(19) United States
(12) Patent Application Publication
(10) Pub. No.: US 2026/0041662 A1
(43) Pub. Date: Feb. 12, 2026

(54) APPLICATION OF TOOSENDANIN IN INHIBITING AURKA KINASE

(71) Applicant: GUANGZHOU MEDICAL UNIVERSITY, Guangzhou, Guangdong (CN)

(72) Inventors: Xiyong Yu, Guangzhou, Guangdong (CN); Feng Yin, Guangzhou, Guangdong (CN); Lixin Zhao, Guangzhou, Guangdong (CN); Ao Shen, Guangzhou, Guangdong (CN); Fangqi Deng, Guangzhou, Guangdong (CN); Jishuo Chang, Guangzhou, Guangdong (CN)

(21) Appl. No.: 18/901,162

(22) Filed: Sep. 30, 2024

(30) Foreign Application Priority Data
Aug. 12, 2024 (CN) 202411104228.5

(51) Int. Cl. A61K 31/352; A61K 45/06

(52) U.S. Cl. CPC A61K 31/352; A61K 45/06

(57) ABSTRACT

The present disclosure provides the application of toosendanin in inhibiting AURKA kinase, which belongs to the technical field of toosendanin application. The present disclosure can significantly reduce the expression of AURKA kinase at the protein level. Through qPCR experiments, Western Blot experiments, and Immunofluorescence experiments, the present disclosure demonstrated that toosendanin can inhibit the expression of AURKA kinase in dose-dependent manner at the protein level by contacting toosendanin with target cells.

### SEQUENCE LISTING

The instant application contains a Sequence Listing with 3 sequence, which has been submitted electronically in XML format and is hereby incorporated herein by reference in its entirety. Said XML copy, created on Sep. 27, 2024, is named QDZJ-US-1-19_SEQ Listings.xml, and is 5.60 kbytes in size.

### CROSS-REFERENCE TO RELATED APPLICATIONS

This application claims to the benefit of priority from Chinese Application No. 202411104228.5 with a filing date of Aug. 12, 2024. The content of the aforementioned applications, including any intervening amendments thereto, are incorporated herein by reference.

### TECHNICAL FIELD

The present disclosure relates to the technical field of toosendanin application, in particular to an application of toosendanin in inhibiting AURKA kinase.

### BACKGROUND

AURKA, or AuroraA kinase, also known as serine/threonine kinase, belongs to the Aurora kinase family. AURKA plays a key role in cell cycle regulation, especially in the process of mitosis. AURKA plays an important role in G2/M conversion of the cell cycle, and its abnormal expression can interfere with the function of checkpoint in mitosis, leading to genetic instability and inducing tumor growth. Therefore, AURKA inhibitors are a promising cancer treatment drug.

At present, almost all existing AURKA inhibitors are ATP competitive inhibitors, which competitively bind to the binding site between ATP and AURKA kinase to inhibit its kinase activity. Most of them belong to multiple inhibitors, and there are few selective inhibitors. These inhibitors may cause non-specific inhibition of other kinases that bind to ATP, leading to side effects. Moreover, due to the heterogeneity and adaptability of tumor cells, long-term use of such inhibitors can easily lead to the emergence of AURKA resistance. In addition, an increasing number of studies have shown that the non kinase activity of AURKA also plays a significant role in cancer, which limits the clinical efficacy of ATP competitive AURKA inhibitors.

Natural compounds, as an important source for drug development, have the advantages of diverse structures, wide biological activity, and relatively small side effects. Therefore, AURKA inhibitors obtained from natural compounds have great potential. By inhibiting the expression of AURKA protein at the transcriptional level, these compounds are expected to block the abnormal activation of AURKA kinase at the source, thereby more effectively inhibiting the proliferation and growth of tumor cells. Compared with traditional ATP competitive inhibitors, this transcriptional level inhibition mechanism may have lower drug-resistance risk and better specificity, bringing new insights and progress to cancer treatment research targeting AURKA.

In this background, the search for new compounds with inhibitory effects on AURKA kinase has important scientific significance and clinical value.

### SUMMARY

The objective of the present disclosure is to provide the application of toosendanin in inhibiting AURKA kinase, wherein toosendanin inhibits the expression of AURKA kinase genes in dose-dependent manner at the protein level, and toosendanin can exert a strong inhibitory effect on AURKA kinase at the nanomolar-scale.

In order to achieve the above disclosure objective, the present disclosure provides the following technical solution:

The present disclosure provides an application of toosendanin in inhibiting AURKA kinase. Preferably, the toosendanin inhibits an expression of AURKA kinase at a protein level.

Preferably, the application comprises: contacting the toosendanin with target cells.

Preferably, the toosendanin is used alone or in combination with other medicinal ingredients.

Preferably, the toosendanin is capable of being made into tablets, capsules, electuaries, drops, granules or injections for use.

Preferably, the application of toosendanin in inhibiting AURKA kinase further comprises using the toosendanin in a preparation of drugs for the prevention and/or treatment of tumors.

The present disclosure further provides a drug for a prevention and/or treatment of tumors, preferably, including an active ingredient toosendanin.

Compared with the existing technology, the present disclosure can inhibit the expression of AURKA kinase genes in dose-dependent manner at the protein level, and the inhibitory effect of toosendanin on AURKA kinase can be strong at the nanomolar-scale.

### BRIEF DESCRIPTION OF THE DRAWINGS

FIG. 1 shows the profile of the AURKA dual luciferase reporter plasmid;

FIG. 2 shows the high-throughput screening results of the dual luciferase assay;

FIG. 3 shows the results of the second round of qPCR experiment screening;

FIG. 4 shows the qPCR dose-response curve;

FIG. 5 shows the experiment results of the Western Blot;

FIG. 6 shows the experimental results of Immunofluorescence.

### DETAILED DESCRIPTION OF THE EMBODIMENTS

The present disclosure provides an application of toosendanin in inhibiting AURKA kinase.

The structural formula of the toosendanin is:

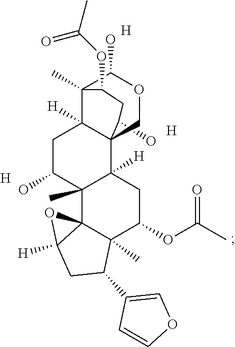

In the present disclosure, the preferred application of toosendanin in inhibiting AURKA kinase is:

The promoter base sequence of human AURKA was retrieved from the NCBI database as shown in SEQ ID NO: 1;

The SEQ ID NO: 1 is:
catgctaagtactttgtaagcatcagttcactgaataaaatcattc
aatgtattcaagtatatgaagtagtacatgaacatagtaggtcta
tgcttgagtagaaaatcctgggacacaaataaagatgatgaaatg
ggttcaaggaggtcaggacacctactcaacgtaaaaagtggctga
gttacttgtccaaggttaaagcacaccagaattgccaactccaga
tccctgagcttaaccactgaaggagttcaggaaatgccagcccaa
aacaagatgctttggcgggctgattaccttgaactgagggcacct
ggagaacagcaggcgggaagaggctttccctgtgctttccttacc
tgcctaaagtctgaccctccaaaaatacctcaattgtcacgaatc
cttataaaccagggaagattaactcggatcacatgagagattaga
ggctgacaccacccagaagatcacctattctgtctgcataacaaa
cttgattcaccatacatttcctccgttcaccgtcccataacttgt
gttagagctgcttcccgccccccgcccccaattctctattcattc
ctttcagtagctcaggatgccatgtaggctttaatcatctgactt
caacttttagtctcgtattttgtgggactcctgtacgtaattaaa
tatcgtttttctcccatggacaatgttcagttacccgaaaaaaat
gggagattcttaagtaccaataaagaatgaaaatcacaagtgggt
agtctggcaaagaaaagttgatgagagattctactgttaacgttg
ggttaatatgactcgaataatttccgtatgctagctcaatgcttt
acatgcacatcctgtttaccctgcagtaatctgttaattcgtctt
atgtgaatttaactcttagtcctaataaggtacctggaaggatgg
agggaagccatttttggttcccctacaccactgttctatccggtc
tcttcacttctgctgagcacatcccgggtgtacatacacacacac
acacacacatatgtgtgtgtatatatatgtatatgtgtgtgtgtg
tgtatatatatatatattttaagtggcccacccctaacttctcaa
ctcccacagaacgttcactcgccaggtaaacagaagcctaattat
ccccaatttgcaggtgagcacacgaggacaagaacccgatccagg
accggatacatcgcagttggaaaggctagaacacagatgccccct
cactatatgcgccgcgaccatctggatgcagaggcgaactaagga
ctgggtgggaatggaagcgaggcccttcgagaagaggaaggggtg
caggccaagccgggcaacttaggaaacgcaaagtagaggcgcatg
ccaccttgctaactctcgactcttccagtctcgccccagtcgttt
ctgtggttttctctaaatgccccagccgaccgcaccagctactct
ccccgtgtcccagtaccagctggtccggttctcttggtatcccgc
tctctcctggaaaaatggaggcgcgaatcctgcccaatctaccgc
tccgagcgcacgttcactgcgcacgctgaaagggcgccaagccga
ccgctgcgctatcgatcggtcccactctctcttgcttttctcgcc
atcttacttactggcacgttcaaaggttagttcacctccttggac
tttatctccaatgcgtcaagcttgacgtcaaggggctgttgcttc
accgataaatggccgaccgcggagagcaccctggggctgggactg
ccacaggtctggctggccgttggctccaccacttccgggttctta
gggagcaagtgcgcctgcgcgcggtgtgcgcccttaaacgcgact
caaggcgtcgggtttgttgtcaaccaatcacaaggcagcctcgct
cgagcgcaggccaatcggctttctagctagagggtttaactccta
tttaaaaagaagaacctttgaattctaacggctgagctcttggaa
gacttgggtccttgggtcgcag.

Design PCR primers for amplifying SEQ ID NO: 1 with MIul and BamHI cleavage sites simultaneously, and the PCR primers include SEQ ID NO: 2 and SEQ ID NO: 3.

The SEQ ID NO: 2 is
cgacgacgcgtcatgctaagtactttgtaagc.
The SEQ ID NO: 3 is
gcgggatcccctcgtccgccactgagatatc.

Taking the whole genome DNA of 293T cells as a template, PCR amplification was performed to obtain PCR amplification products. The synthesis of PCR amplification products was proved by agarose gel electrophoresis.

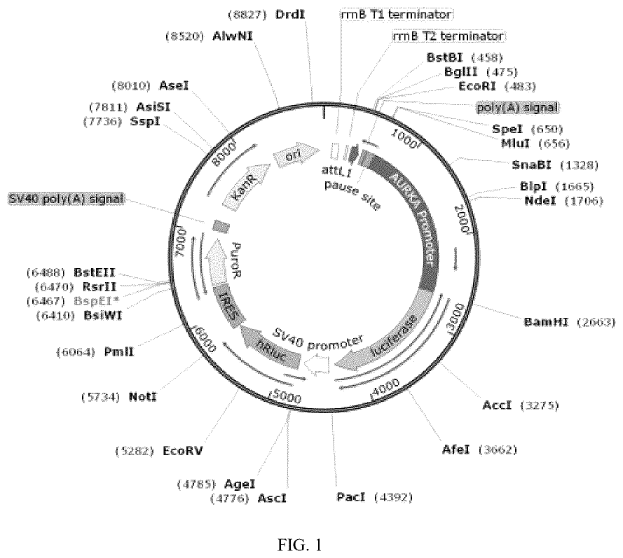

The PCR amplification product was inserted into the dual luciferase reporter vector through two enzyme cleavage sites, MIul and BamH1, and the successful construction of the AURKA dual luciferase reporter plasmid was demonstrated by sequencing, as shown in FIG. 1.

The dual luciferase reporter vector described in the present disclosure was purchased from Beijing Tsingke Biotech Co., Ltd., and the sequencing can be performed using techniques that are well-known to those skilled in the art.

293T cells in logarithmic growth phase were spreaded evenly in a 10 cm medium dish at a quantity of 4×10⁶ cells/ml, culturing for 24 hours in a 37° C., 5% CO₂ cell culture incubator.

Configure transfection system to conduct transfection experiments. The transfection system includes A tube and B tube, wherein A tube includes 500 μM opti-mem and 24 μM PEI, and B tube includes 500 μM opti-mem and 12 μM AURKA dual luciferase reporter plasmid. The A tube and B tube were mixed evenly and incubated at room temperature for 15 minutes. After 15 minutes, the transfection system was uniformly added to 293T cells and subjected to medium exchange treatment after 8 hours.

After 24 hours, the 293T were digested, spreading the 293T transfected with AURKA dual fluorescent reporter plasmid at a density of 1× 10⁴ cells per well in a 96-well plate, culturing in a 37° C., 5% CO2 cell culture incubator for 12 hours.

After 12 hours of cultivation, 10 μM of the compound according to the recommended screening dose of natural compound library L6000 was added, and the natural compound library L6000 was purchased from Shanghai Topscience Co., Ltd.

After 24 hours, cells were lysed and high-throughput screening of drugs was performed using dual luciferase assay, specifically:
(1) Remove the cell culture medium, and wash the cells twice with PBS, and then adding 50 μL of cell lysis buffer to each well and lyse on ice for 5 minutes;
(2) After complete lysis, taking 20 μL of lysis buffer to add it to the enzyme-linked immunosorbent assay plate;
(3) Prepare firefly luciferase reaction solution and renilla luciferase reaction solution, that is, firefly luciferase substrate (50×) and renilla luciferase substrate (50×) were diluted with corresponding buffer solutions to 1× working solution, and incubated at room temperature;
(4) Add 100 μL of firefly luciferase reaction solution to the enzyme-linked immunosorbent assay plate, mix well on the plate, and detecting the activity of firefly luciferase;
(5) Add 100 μL of renilla luciferase reaction solution, shaking the plate for mixing, and detect the activity of renilla luciferase.

According to the dual luciferase reporter assay, the final experimental results were calculated using the following method: Experimental group ratio=(Experimental group F-Background F)/(Experimental group R-Background R); Control group ratio=(Control group F-Background F)/(Control group R-Background R); and Multiple of expression=Experimental group ratio/Control group ratio.

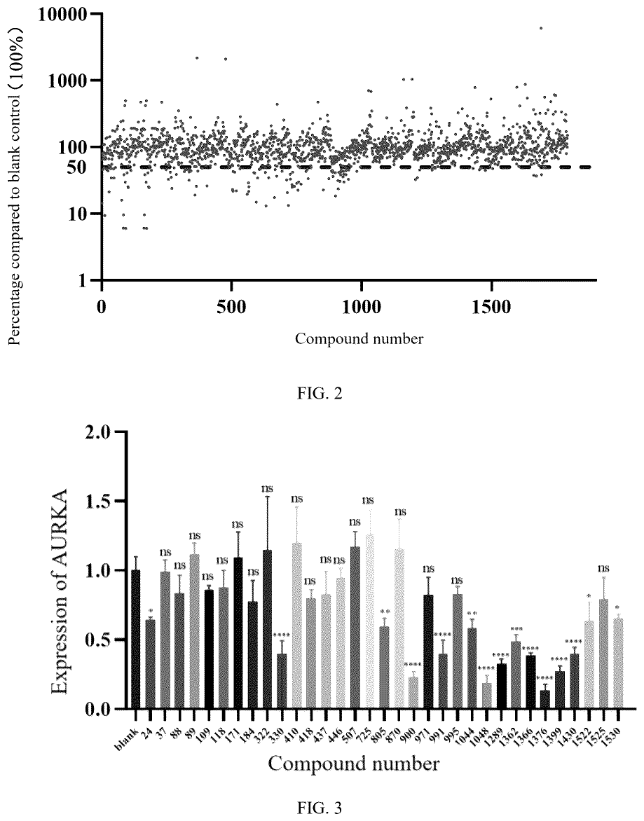

Finally, 36 candidate compounds capable of inhibiting AURKA expression at the transcriptional level were screened through dual luciferase assay, as shown in FIG. 2.

A second round of screening was performed through qPCR experiments:
(1) Inoculate PC9 lung cancer cells in logarithmic growth phase at a density of 2.5×10⁵ cells per well into a 12-well plate, and culture for 24 hours in a 37° C., 5% CO₂ cell culture incubator;
(2) After 24 hours, the 36 compounds obtained from the above screening were added to the cell culture medium at a concentration of 10 μM, and cultured in a 37° C., 5% CO₂ cell culture incubator for 24 hours;
(3) After 24 hours, remove the cell plate from the incubator, remove the culture medium, and wash the cells twice with PBS;
(4) Extract RNA according to the instructions of the RNA extraction kit, specifically: add 500 μL of lysis buffer to each well, repeatedly blow and beat the lysed cells, place them at room temperature for 5 minutes; after complete lysis, take all the lysis buffer and add it to a 1.5 mL centrifuge tube; add an equal volume of anhydrous ethanol, blow and mix well to obtain a mixture; take 600 μL of mixed solution and add it to the RNA purification column; centrifuge at 12000r for 1 minute, then remove the liquid in the collection tube and add 500 μL of wash buffer; centrifuge at 12000r for 1 minute; remove the liquid in the collection tube, centrifuge again at 12000r for 1 minute, and open the lid for 2 minutes to dry; transfer the adsorption column to a new 1.5 mL centrifuge tube, add 30 μL of DEPC water dropwise onto the film, and centrifuge at 12000r for 1 minute;
(5) Open the Nano Drop ONE ultramicro ultraviolet spectrophotometer, select “RNA” on the “Nucleic Acid tab” page, measure the concentration of the liquid in the collection tube in sequence, and record the concentration;
(6) Take an 8-tube strip and add DEPC water, RNA, and DNAase (5×) in sequence, wherein the total mass of the system is 800 ng and the total volume is 15 μL; open the MiniAmp Plus PCR thermal cycler, place the 8-tube strip into the PCR thermal cycler, select the set method, run the PCR thermal cycler, and after incubation was complete, add 5 μL of reverse transcription reagent and select the set method for incubation;
(7) Each group was prepared with three replicates, each containing 5 μL of SYBR Green, 3.2 μL of LDEPC water, 1 μL of cDNA, and 0.8 μL of mixed primers; load the sample after the solution was prepared; after covering the film, centrifuge and turn on the real-time fluorescence quantitative PCR instrument (Roche); open the admin software, select the set method, place the well plate into the instrument, and click for running.

Finally, the final experimental results were calculated according to the qPCR experiment using the following method: ΔCT=CT value of the target gene−mean CT value of the reference gene; ΔΔCT=Experimental group ΔCT−mean of Control group ΔCT, calculate the 2-^{ΔΔCT} value.

Finally, 10 traditional Chinese medicine monomer compounds were obtained that can inhibit AURKA expression at the gene transcription level, as shown in FIG. 3. This includes compound 330:10-hydroxydecanoic acid, compound 900: γ-mangostin, compound 991: icaritin, compound 1048: Trilobatin, compound 1289: Flavokawain A, compound 1362: cinnamyl acetate, compound 1366: palmitic acid, compound 1376: toosendanin, compound, 1399:7-demethylsuberosin, and compound 1430:7-hydroxycoumarin.

The structural formula of compound 330, 10-hydroxydecanoic acid, is:

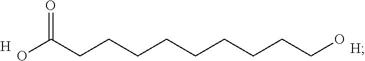

The structural formula of compound 900, γ-mangostin, is:

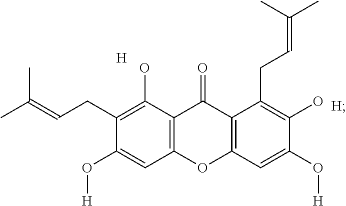

The structural formula of compound 991, icaritin, is:

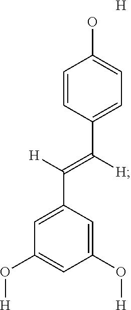

The structural formula of compound 1048, Trilobatin, is:

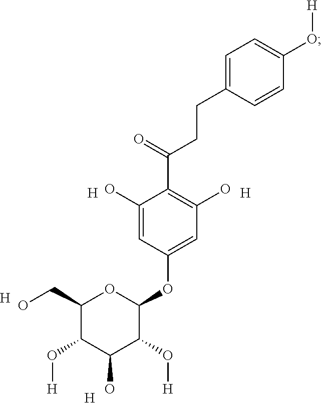

The structural formula of compound 1289, Flavokawain A, is:

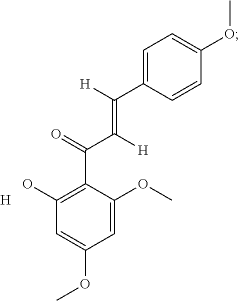

The structural formula of compound 1362, cinnamyl acetate, is:

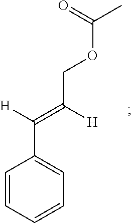

The structural formula of compound 1366, palmitic acid, is:

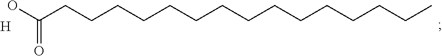

The structural formula of compound 1399, 7-demethylsuberosin, is:

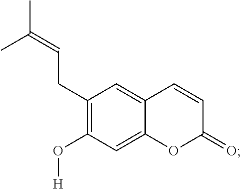 [SMILES: CC(C)=CCc1cc2ccc(=O)oc2cc1O]

The structural formula of compound 1430, 7-hydroxycoumarin, is:

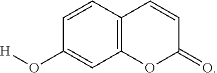 [SMILES: O=c1ccc2ccc(O)cc2o1]

The Half-effective concentration of toosendanin was detected by qPCR experiment, specifically:

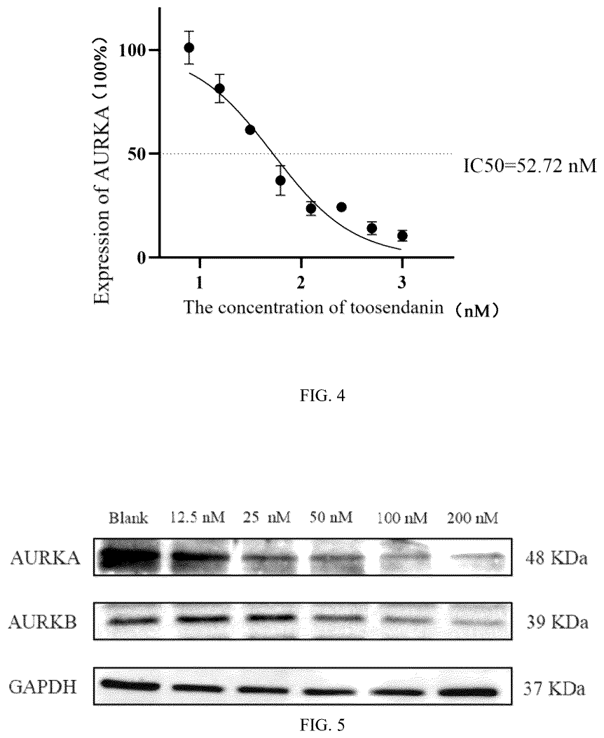

PC9 lung cancer cells were seeded into a 12-well plate at a density of 2.5×10⁵ cells per well. After 24 hours of culture, the seeded cells were added with different concentrations of 10 μM, 5 μ, 2.5μ, 1.25μ, 0.625μ, 0.3125μ, 0.15625μ, and 0.078125μ M of toosendanin for 24 hours culture. qPCR experiments were performed according to the above steps, and the half-effective concentration of toosendanin for inhibiting AURKA gene expression was 52.72 nM, as shown in FIG. 4.

Based on the above, it can be seen that the toosendanin inhibits an expression of AURKA kinase at the protein level.

In the present disclosure, the toosendanin is used alone or in combination with other medicinal ingredients.

In the present disclosure, the toosendanin is capable of being made into tablets, capsules, electuaries, drops, granules or injections for use.

In the present disclosure, the application of toosendanin in inhibiting AURKA kinase further includes using the toosendanin in the preparation of drugs for the prevention and/or treatment of tumors.

The present disclosure further provides a drug for a prevention and/or treatment of tumors, preferably, including an active ingredient toosendanin.

In the present disclosure, unless otherwise specified, the required instruments or technical means may be those familiar to those skilled in the art.

The following provides a detailed description of the technical solutions provided by the present disclosure in conjunction with the embodiments, but the embodiments should not be regarded as limiting the scope of the present disclosure.

### Embodiment 1

Western Blot assay was used to detect the inhibition effect of toosendanin on AURKA expression at protein level.

1. Inoculate PC9 lung cancer cells in logarithmic growth phase at a density of 2.5×10⁵ cells per well into a 6-well plate and culture for 24 hours in a cell culture incubator at 37° C. and 5% CO₂;

2. Treat with different concentrations of toosendanin, and dilute the experimental drugs with DMSO; set the drug concentration to 12.5 nM, 25 nM, 50 nM, 100 nM, and 200 nM, culturing in a cell culture incubator at 37° C. and 5% CO₂;

3. Protein extraction:
(1) Remove the cell culture medium, wash the cells twice with PBS, then add 100 μL of cell lysis buffer to each well, and lyse on ice for 10 minutes;
(2) After complete lysis, take the lysis buffer and add it to a 1.5 mL centrifuge tube;
(3) Insert the centrifuge tube into ice, and ultrasound followed by vortex is performed three times for 10 minutes each time;
(4) Place the centrifuge tube into a pre-cooled centrifuge, then perform centrifuge at a temperature of 4° C. and a speed of 12000r for 10 minutes, and then transfer the supernatant to a new 1.5 mL centrifuge tube;
(5) Measure the protein concentration using the BCA method, with a sample size of 30 μg;
(6) Add loading buffer (5×) at a ratio of 4:1, vortex thoroughly and mix well, then boil in a water bath at 100° C. for 5 minutes, and then cool on ice.

4. Arrange the prefabricated film (from Beyotime Biotech Inc.) onto the electrophoresis accessory, pour the electrolytic powder into 1 L of pure water and mix well, pour the electrolyte into the electrophoresis tank, and slowly pull out the comb vertically;

5. Add 20 μL of loading buffer, 4 μL of marker, 20 μL of protein sample, 4 μL of marker, and 4 μL of marker in the sample wells from left to right, then pour half a box of electrolyte into the outer box, connect to the power supply, set to 80V constant voltage, and run for 90 minutes;

6. Soak the clamp plate, sponge, and cotton in the frozen transfer buffer, remove the prefabricated agar from the box, use a scraper to cut off the excess four sides, place the agar on the cotton in the transfer buffer, cover the PVDF film soaked in methanol on the agar, drive away bubbles, cover with cotton and sponge, and then cover the clamp plate;

7. Place the clamp plate into the component of the instrument, put it into the box and fill it with the transfer buffer; put it into the ice box, place the box inside the ice, connect to the power supply, set the 100V constant current, and run for 120 minutes;

8. Remove the clamp plate, use a clip to remove the PVDF film and place it in a clean and dry empty box; take 2 g of skimmed milk powder (sourced from Beyotime Biotech Inc.) to add to 40 ml TBST, then pour the prepared skimmed milk into the box and place the box on a shaking table for 2 hours;

9. Remove skim milk, add TBST, and shake on a 120r shaker for 5 minutes for cleaning, and repeat this process three times, then take the film out for cutting; place the cut film in different wells, then pour GAPDH (1:1000), AURKA (1:1000), and AURKB (1:1000) antibodies into them, and place them in an ice box at a temperature of 4° C. overnight;

10. Retrieve the primary antibody, wash three times with TBST, then pour in the secondary antibody, and incubate it on a 40r shaker for 2 hours;

11. Retrieve the secondary antibody, wash three times with TBST, place the film on the instrument for exposure, use ImageJ software to calculate the grayscale value of the corresponding band, and perform statistical analysis on the absorbance ratio with the internal reference GAPDH, as shown in FIG. 5.

### Embodiment 2

Immunofluorescence assay was performed to detect the inhibition effect of toosendanin on AURKA expression at protein level.

1. Inoculate PC9 lung cancer cells in logarithmic growth phase at a density of 1×10⁵ cells per well into a confocal dish and culture for 24 hours in a cell culture incubator at 37° C. and 5% CO₂;

2. Treat with different concentrations of toosendanin, and dilute the experimental drugs with DMSO at concentrations of 25 nM, 50 nM, and 100 nM, then, culture the cells in a cell culture incubator at 37° C. and 5% CO₂;

3. Remove the culture medium, and wash three times with PBS; add 400 μL of frozen 4% paraformaldehyde to each well and fix it in a refrigerator at 4° C. for 20 minutes; after rewarming for 5 minutes, remove the waste liquid and wash it three times with PBS;

4. Add 400 μL of 0.5% Triton-100 (1:200) freshly prepared in PBS to each well, incubate at room temperature for 20 minutes, and wash three times with PBS;

5. Add 400 μL of 5% blocked goat serum (1:20) prepared with PBS to each well, then block them at room temperature for 1 hour, and wash three times with PBS;

6. Add 400 μL of primary antibody to each well and place them overnight in a refrigerator at a temperature of 4° C.;

7. Rewarm the sample for 30 minutes, retrieve the primary antibody, and wash three times with PBS;

8. Pour in the secondary antibody, and place it on a shaker at 37° C. for 1 hour;

9. Remove the secondary antibody, and wash three times with PBS;

10. Add FITC, incubate at 37° C. for 30 minutes, remove FITC and wash three times with PBS;

11. Add DAPI, incubate at 37° C. for 10 minutes, remove FITC and wash three times with PBS;

12. Add 2 drops of 10% glycerol, and place overnight in a refrigerator at a temperature of 4° C.;

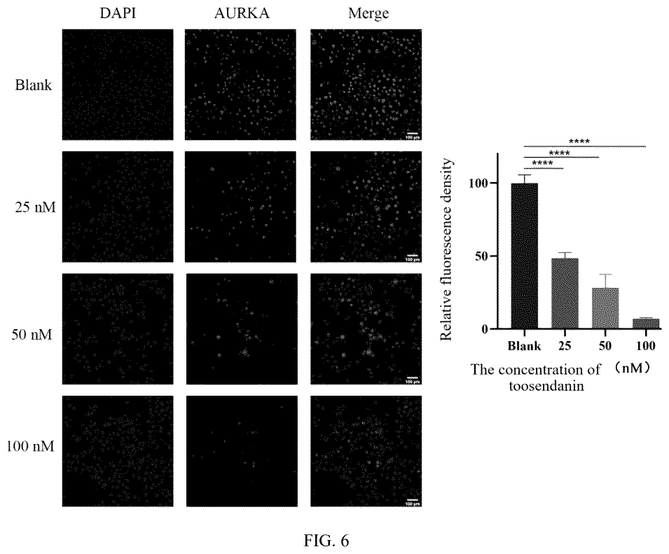

13. Place the confocal dish under a confocal microscope for photography, and perform fluorescence quantitative analysis using ImageJ software fluorescence images, as shown in FIG. 6.

### CONCLUSION

(1) In Embodiment 1, Western Blot experiment was used to detect the inhibition effect of toosendanin on AURKA expression in protein level, and the detection results are shown in FIG. 5. Based on FIG. 5, it can be seen that toosendanin can inhibit the expression of AURKA in dose-dependent manner at the protein level.

(2) In Embodiment 2, Immunofluorescence experiment was conducted to detect the inhibition effect of toosendanin on AURKA expression in protein level, and the detection results are shown in FIG. 6. Based on FIG. 6, it can be seen that toosendanin can inhibit the expression of AURKA in dose-dependent manner at the protein level.

The above is only the preferred embodiment of the present disclosure. It should be pointed out that for ordinary skilled person in the art, several improvements and embellishments can be made without departing from the principles of the present disclosure, and these improvements and embellishments should also be considered as the scope of the present disclosure.

1. An application of toosendanin in inhibiting AURKA kinase.

2. The application of toosendanin in inhibiting AURKA kinase according to claim 1, wherein the toosendanin inhibits an expression of AURKA kinase at a protein level.

3. The application of toosendanin in inhibiting AURKA kinase according to claim 1, wherein the application comprises: contacting the toosendanin with target cells.

4. The application of toosendanin in inhibiting AURKA kinase according to claim 1, wherein the toosendanin is used alone or in combination with other medicinal ingredients.

5. The application of toosendanin in inhibiting AURKA kinase according to claim 1, wherein the toosendanin is capable of being made into tablets, capsules, electuaries, drops, granules or injections for use.

6. The application of toosendanin in inhibiting AURKA kinase according to claim 1, wherein the application of toosendanin in inhibiting AURKA kinase further comprises using the toosendanin in a preparation of drugs for the prevention and/or treatment of tumors.

7. A drug for a prevention and/or treatment of tumors, characterized by comprising an active ingredient toosendanin.


SEQUENCE LISTING (3 sequences)

SEQ ID NO: 1 - DNA, 2048 bp, synthetic construct
  source 1..2048: mol_type = other DNA
catgctaagtactttgtaagcatcagttcactgaataaaatcattcaatgtattcaagtatatgaagtagtacatgaacatagtaggtctatgcttgagtagaaaatcctgggacacaaataaagatgatgaaatgggttcaaggaggtcaggacacctactcaacgtaaaaagtggctgagttacttgtccaaggttaaagcacaccagaattgccaactccagatccctgagcttaaccactgaaggagttcaggaaatgccagcccaaaacaagatgctttggcgggctgattaccttgaactgagggcacctggagaacagcaggcgggaagaggctttccctgtgctttccttacctgcctaaagtctgaccctccaaaaatacctcaattgtcacgaatccttataaaccagggaagattaactcggatcacatgagagattagaggctgacaccacccagaagatcacctattctgtctgcataacaaacttgattcaccatacatttcctccgttcaccgtcccataacttgtgttagagctgcttcccgccccccgcccccaattctctattcattcctttcagtagctcaggatgccatgtaggctttaatcatctgacttcaacttttagtctcgtattttgtgggactcctgtacgtaattaaatatcgtttttctcccatggacaatgttcagttacccgaaaaaaatgggagattcttaagtaccaataaagaatgaaaatcacaagtgggtagtctggcaaagaaaagttgatgagagattctactgttaacgttgggttaatatgactcgaataatttccgtatgctagctcaatgctttacatgcacatcctgtttaccctgcagtaatctgttaattcgtcttatgtgaatttaactcttagtcctaataaggtacctggaaggatggagggaagccatttttggttcccctacaccactgttctatccggtctcttcacttctgctgagcacatcccgggtgtacatacacacacacacacacacatatgtgtgtgtatatatatgtatatgtgtgtgtgtgtgtatatatatatatattttaagtggcccacccctaacttctcaactcccacagaacgttcactcgccaggtaaacagaagcctaattatccccaatttgcaggtgagcacacgaggacaagaacccgatccaggaccggatacatcgcagttggaaaggctagaacacagatgccccctcactatatgcgccgcgaccatctggatgcagaggcgaactaaggactgggtgggaatggaagcgaggcccttcgagaagaggaaggggtgcaggccaagccgggcaacttaggaaacgcaaagtagaggcgcatgccaccttgctaactctcgactcttccagtctcgccccagtcgtttctgtggttttctctaaatgccccagccgaccgcaccagctactctccccgtgtcccagtaccagctggtccggttctcttggtatcccgctctctcctggaaaaatggaggcgcgaatcctgcccaatctaccgctccgagcgcacgttcactgcgcacgctgaaagggcgccaagccgaccgctgcgctatcgatcggtcccactctctcttgcttttctcgccatcttacttactggcacgttcaaaggttagttcacctccttggactttatctccaatgcgtcaagcttgacgtcaaggggctgttgcttcaccgataaatggccgaccgcggagagcaccctggggctgggactgccacaggtctggctggccgttggctccaccacttccgggttcttagggagcaagtgcgcctgcgcgcggtgtgcgcccttaaacgcgactcaaggcgtcgggtttgttgtcaaccaatcacaaggcagcctcgctcgagcgcaggccaatcggctttctagctagagggtttaactcctatttaaaaagaagaacctttgaattctaacggctgagctcttggaagacttgggtccttgggtcgcag

SEQ ID NO: 2 - DNA, 32 bp, synthetic construct
  source 1..32: mol_type = other DNA
cgacgacgcgtcatgctaagtactttgtaagc

SEQ ID NO: 3 - DNA, 31 bp, synthetic construct
  source 1..31: mol_type = other DNA
gcgggatcccctcgtccgccactgagatatc